(19) United States
(12) Patent Application Publication
(10) Pub. No.: US 2025/0288230 A1
(43) Pub. Date: Sep. 18, 2025

(54) MEASUREMENT APPARATUS AND DIELECTRIC CONSTANT MEASUREMENT APPARATUS

(71) Applicant: Taiyo Yuden Co., Ltd., Tokyo (JP)

(72) Inventors: Takayuki SEKIGUCHI, Tokyo (JP); Katsuhiro OYAMA, TOKYO (JP)

(21) Appl. No.: 19/220,441

(22) Filed: May 28, 2025

Related U.S. Application Data
(63) Continuation of application No. PCT/JP2023/025296, filed on Jul. 7, 2023.

(30) Foreign Application Priority Data
Nov. 30, 2022 (JP) 2022-192448

(51) Int. Cl. A61B 5/1477; A61B 5/00; A61B 5/145

(52) U.S. Cl. CPC A61B 5/1477; A61B 5/14532; A61B 5/14546; A61B 5/681

(57) ABSTRACT

A measurement apparatus includes a substrate that is a dielectric, a ground conductor provided on the substrate, a first signal line provided separately from the ground conductor on the substrate and against which a living body is pressed, a second signal line provided on the substrate such that the second signal line is separated from the ground conductor and the first signal line, and the second signal line does not come into contact with the living body when the living body is pressed against the first signal line, an oscillation circuit that oscillates an alternating current first signal, and a computation circuit that acquires biological information according to comparison of a second signal that is the first signal passing through the first signal line and a third signal that is the first signal passing through the second signal line.

### CROSS-REFERENCE TO RELATED APPLICATIONS

This application is a continuation of PCT International Application No. PCT/JP 2023/025296 filed on Jul. 7, 2023, which designates the United States, incorporated herein by reference, and which claims the benefit of priority from Japanese Patent Application No. 2022-192448, filed on Nov. 30, 2022, incorporated herein by reference.

### FIELD

The present embodiment relates to a measurement apparatus and a dielectric constant measurement apparatus.

### BACKGROUND

Some pieces of biological information, such as blood glucose level, are measured by using means, such as blood collection, that is invasive for the body. However, to alleviate the physical burden of the subject or reduce the risk of contraction of an infectious disease, a technique that can measure the biological information as accurately as possible with a non-invasive method is demanded in recent years.

A related technique is described in Japanese Translation of PCT International Application Publication No. 2021-502880.

There are needs to provide a measurement apparatus and a dielectric constant measurement apparatus that can non-invasively and highly accurately measure biological information.

### SUMMARY

According to an aspect of the present invention, a measurement apparatus includes a substrate that is a dielectric, a ground conductor provided on the substrate, a first signal line provided separately from the ground conductor on the substrate and against which a living body is pressed, a second signal line provided on the substrate such that the second signal line is separated from the ground conductor and the first signal line, and the second signal line does not come into contact with the living body when the living body is pressed against the first signal line, an oscillation circuit that oscillates an alternating current first signal, and a computation circuit that acquires biological information according to comparison of a second signal that is the first signal passing through the first signal line and a third signal that is the first signal passing through the second signal line.

### BRIEF DESCRIPTION OF THE DRAWINGS

FIG. 1 is a diagram illustrating complex dielectric constants of a plurality of aqueous solutions with different glucose concentrations;

FIG. 2 is a diagram of a subject wearing a blood glucose level measurement apparatus according to a first embodiment;

FIG. 3 is a cross-sectional view of the blood glucose level measurement apparatus according to the first embodiment illustrated in FIG. 2, cut along a cutting line III-III;

FIG. 4 is an external view of the blood glucose level measurement apparatus according to the first embodiment as viewed from a lower surface;

FIG. 5 is a schematic diagram illustrating a configuration of the blood glucose level measurement apparatus according to the first embodiment;

FIG. 6 is a diagram of a sensor according to the first embodiment as viewed from a +Z direction;

FIG. 7 is a diagram of the sensor according to the first embodiment as viewed from a −Z direction;

FIG. 8 is a diagram of a cross section of the sensor according to the first embodiment illustrated in FIG. 6, cut along a cutting line VIII-VIII;

FIG. 9 is a diagram of a cross section of the sensor according to the first embodiment illustrated in FIG. 6, cut along a cutting line IX-IX;

FIG. 10 is a schematic diagram illustrating an electromagnetic field distribution when a skin of the subject is pressed against a first signal line according to the first embodiment;

FIGS. 11A and 11B depict schematic diagrams describing changes in the phase of an alternating current (AC) signal passing through the first signal line when the skin of the subject is pressed against the first signal line of the sensor according to the first embodiment in a case where the stomach of the subject is empty and in a case where the subject has eaten a meal;

FIG. 12 is a diagram describing application of pressure to the sensor according to the first embodiment when the subject tightens a band;

FIG. 13 is a diagram illustrating that the phase of the AC signal at an output end of the first signal line changes according to the pressure;

FIG. 14 is a diagram illustrating a positional relation between the first signal line and a second signal line in the sensor according to the first embodiment;

FIG. 15 is a diagram describing a time shift of a phase lead Rd2 when the skin of the subject is pressed against the first signal line in the sensor according to the first embodiment;

FIG. 16 is a flow chart illustrating an example of an operation of the blood glucose level measurement apparatus according to the first embodiment;

FIG. 17 is a schematic diagram illustrating an example of a configuration of a blood glucose level measurement apparatus according to a second embodiment;

FIG. 18 is a cross-sectional view of a sensor according to a first modification cut along an XZ plane;

FIG. 19 is a cross-sectional view of a sensor according to a second modification cut along the XZ plane;

FIG. 20 is a cross-sectional view of a sensor according to a third modification cut along the XZ plane; and

FIG. 21 is a diagram depicting a blood glucose level measurement apparatus according to a fourth modification as well as a band.

### DETAILED DESCRIPTION

It is known that a concentration of glucose included in interstitial fluid of a dermis layer is correlated with a concentration of glucose in blood, that is, a blood glucose level.

Moreover, a dielectric constant of a liquid changes depending on a concentration of glucose included in the liquid.

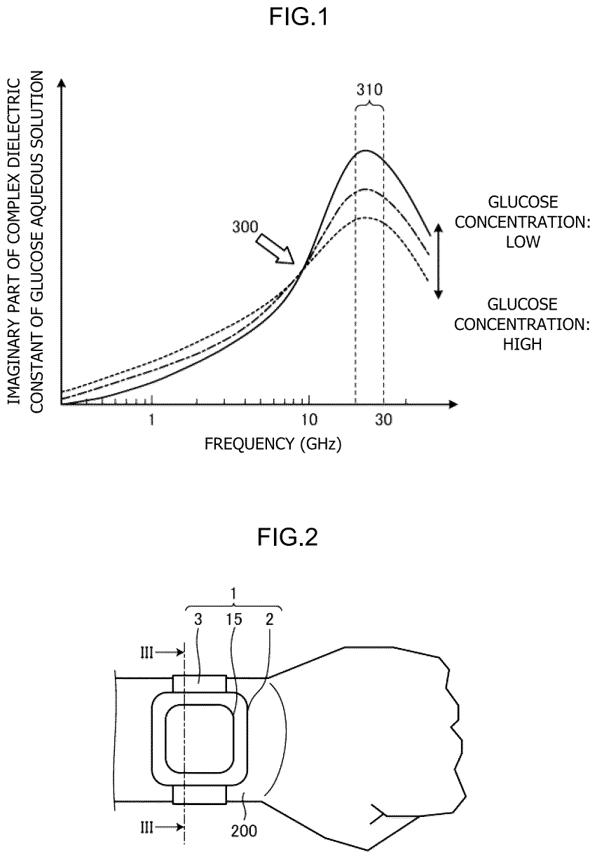

FIG. 1 is a diagram illustrating complex dielectric constants of a plurality of aqueous solutions with different glucose concentrations. In FIG. 1, a vertical axis represents an imaginary part of the complex dielectric constant, and a horizontal axis represents a frequency.

As illustrated in FIG. 1, the imaginary part of the complex dielectric constant (hereinafter, the imaginary part of the complex dielectric constant will simply be referred to as an imaginary part) exhibits different frequency characteristics according to the glucose concentration. Note that, as indicated by arrows on the right side of the graph, the glucose concentration becomes higher toward the lower side, and the glucose concentration becomes lower toward the upper side.

As illustrated in FIG. 1, at frequencies higher than an inflection point 300 existing near 10 GHz, the higher the glucose concentration in the aqueous solution, the smaller the imaginary part. Moreover, the intervals of three curves are wide in a frequency range 310, and the imaginary part largely changes with respect to a change in the concentration of glucose.

Note that a real part of the complex dielectric constant (hereinafter, the real part of the complex dielectric constant will simply be referred to as a real part) changes with the opposite tendency to the imaginary part. That is, at frequencies higher than the inflection point 300, the higher the glucose concentration in the aqueous solution, the larger the real part. The dependency of the real part on the glucose concentration is large in the frequency range 310, and this is similar to the imaginary part.

Hereinafter, the dielectric constant denotes the real part of the complex dielectric constant.

The dependency of the dielectric constant of the skin of the human body, specifically, the dermis layer, is similar to the glucose concentration dependency illustrated in FIG. 1 with respect to the glucose concentration in the interstitial fluid of the dermis layer. Moreover, the glucose concentration in the interstitial fluid of the dermis layer and the blood glucose level are correlated as described above. Hence, if the value of the dielectric constant of the skin can be obtained, the blood glucose level can be estimated. A measurement apparatus of embodiments estimates the blood glucose level on the basis of the value of the dielectric constant of the skin.

In the embodiments, a sensor with a structure of a transmission line provided with a signal line on a substrate is used as a sensor that acquires the value of the dielectric constant of the skin. An AC signal flows through the signal line, and when a subject touches the signal line, the wavelength of the AC signal flowing through the signal line changes according to the dielectric constant of the skin touching the signal line. The change in the wavelength is related to the dielectric constant of the skin. The measurement apparatus of the embodiments can measure the change in the wavelength of the AC signal flowing through the signal line to determine and acquire the blood glucose level.

After all, the blood glucose level can be measured just by the subject touching the signal line, and non-invasive blood glucose level measurement can be realized. Note that non-invasive here means not hurting the living body. That is, non-invasive means that the measurement is possible just by the touch.

Note that the measurement apparatus of the embodiments can be implemented in any apparatus or electronic equipment. The measurement apparatus can be implemented in a wearable apparatus such as a smartwatch. Note that the measurement apparatus of the embodiments may be a stationary measurement apparatus.

In addition, the biological information obtained by the measurement apparatus of the embodiments is not limited to the blood glucose level. The variation of the measurement target will be described later.

Hereinafter, a blood glucose level measurement apparatus will be described with reference to the drawings. Note that the present invention is not limited to the embodiments described below.

### First Embodiment

FIG. 2 is a diagram of the subject wearing the blood glucose level measurement apparatus.

A blood glucose level measurement apparatus 1 includes a case 2 with a flat shape, a display apparatus 15 attached to the surface of the case 2, and a band 3 attached to the side surface of the case. The structure is the same as the structure of a commercially available watch or smartwatch.

The case 2 includes an upper surface, a lower surface, and side surfaces connecting the periphery of the upper surface and the periphery of the lower surface.

The band 3 is attached to one side surface and another side surface of the case 2. When the band 3 is wound around an arm 200 of the subject, the lower surface of the case 2 is brought into contact with and fixed to the arm 200. The blood glucose level measurement apparatus 1 outputs various types of image information to the display apparatus 15. The subject can visually check various types of image or display information output to the display apparatus 15 and can check the measurement result of the blood glucose level here.

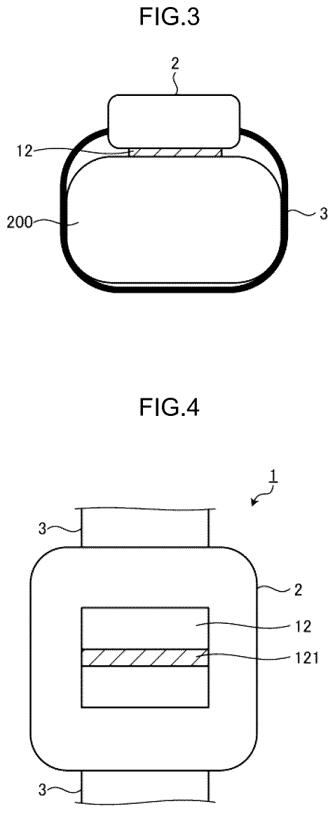

FIG. 3 is a cross-sectional view cut along a cutting line III-III of FIG. 2. FIG. 4 is an external view of the blood glucose level measurement apparatus 1 as viewed from the lower surface.

As illustrated in FIGS. 3 and 4, a sensor 12 is provided on the lower surface of the case 2.

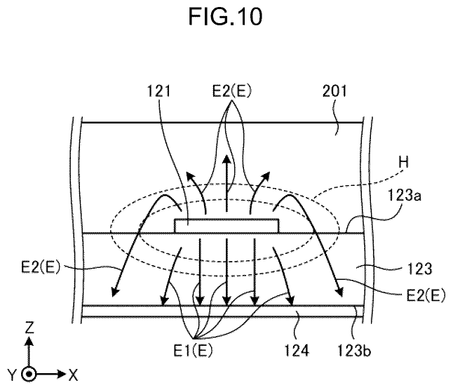

The sensor 12 includes a substrate 123, a ground conductor 124 provided without a gap on one surface of the substrate 123, and a first signal line 121 provided on the other surface. See FIG. 10.

Note that, in the example, the first signal line 121 is provided in a straight line shape (strip line) in the same direction as the direction of the extended arm of the subject wearing the apparatus. The first signal line 121 is provided in such a manner as to come into contact with the skin of the wearer.

Note that the first signal line 121 may be covered by a thin insulating film.

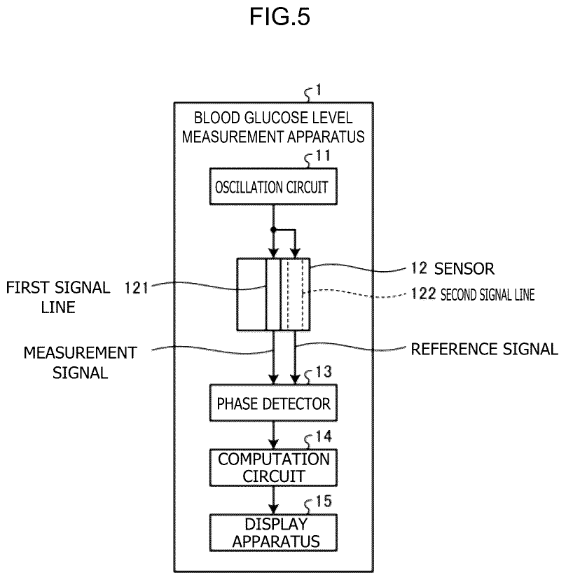

FIG. 5 is a schematic view illustrating a configuration of the blood glucose level measurement apparatus 1. As illustrated in FIG. 5, the blood glucose level measurement apparatus 1 includes at least an oscillation circuit 11, the sensor 12, a phase detector 13, a computation circuit 14, and the display apparatus 15.

In a first embodiment, the sensor 12 has a structure of a microstrip line that is a type of transmission line.

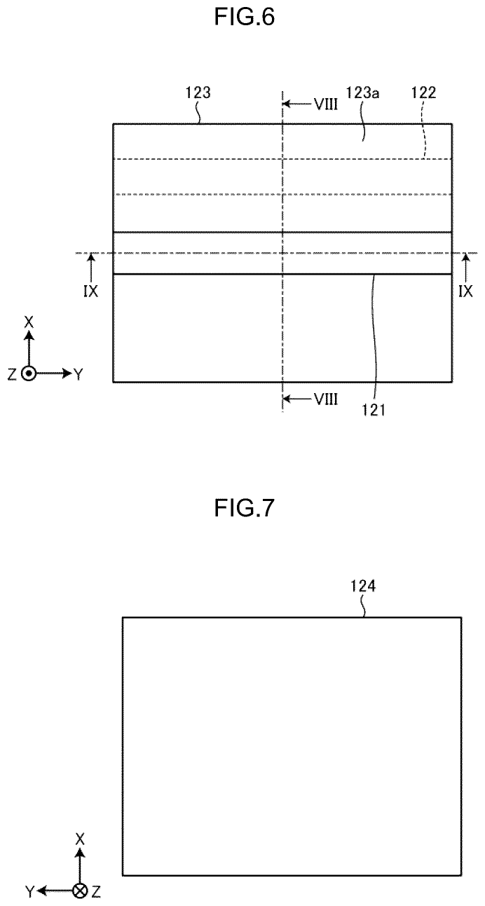

The sensor 12 will be described below with reference to FIGS. 6 to 9.

In FIGS. 6 to 9 and subsequent figures, the direction in which the sensor 12 comes into contact with the skin of the subject will be referred to as a +Z direction, the direction of the extension of the first signal line 121 that is orthogonal to the +Z direction will be referred to as a +Y direction, and the direction orthogonal to the +Z direction and the +Y direction will be referred to as a +X direction to describe the sensor 12.

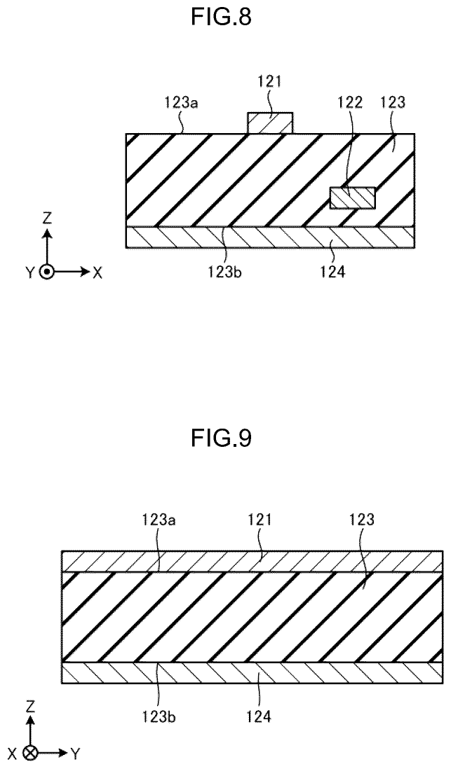

FIG. 6 is a diagram of the sensor 12 as viewed from the +Z direction (surface side abutted to the skin). FIG. 7 is a diagram of the sensor 12 of the first embodiment as viewed from the −Z direction (surface side attached to the case). FIG. 8 is a diagram of a cross section cut along a cutting line VIII-VIII of FIG. 6. FIG. 9 is a diagram of a cross section cut along a cutting line IX-IX of FIG. 6.

The sensor 12 includes the substrate 123 including a dielectric. The material of the substrate 123 can be a general substrate material such as polytetrafluoroethylene (PTFE) and polyimide. Note that the substrate 123 has a rectangular flat shape.

In FIG. 6, the first signal line 121 including a conductor is provided on a surface 123a of the substrate 123 to be abutted to the skin. The first signal line 121 extends in the Y direction (left and right) through substantially the center part in plan view.

To implement the first signal line 121 on a printed board, a plating process or a foil pasting technique is used to pattern the first signal line 121 through a photo etching process. Hence, the width and the thickness of the first signal line 121 are constant.

The ground conductor 124 is formed across the entire surface of a surface 123b opposite to the surface provided with the first signal line 121. The signal lines 121 and 124 include a material with high electrical conductivity such as copper and gold.

Note that the surface 123a of the substrate 123 is referred to as a first surface, and the surface 123b is referred to as a second surface that is a surface on the opposite side of the first surface.

The first signal line 121 is pressed against a skin 201 of the wearer as illustrated in FIG. 10 in a state in which an AC electrical signal is applied to the first signal line 121.

Here, FIG. 10 is a schematic diagram illustrating an electromagnetic field distribution. Arrows E represent the electrical field vector, and a dotted line H represents the magnetic field distribution.

When the AC signal flows through the first signal line 121, the electric field vector E is formed around the first signal line 121. Most electric field vector E1 is concentrated between the first signal line 121 and the ground conductor 124, but there is some electric field vector E2 coming out of the substrate 123 from the surface 123a. When the skin 201 is in contact with the first signal line 121, the electric field vector E2 passes through the skin 201, and the wavelength of the AC signal flowing through the first signal line 121 changes.

Hereinafter, the change in the wavelength of the AC signal flowing through the first signal line 121 will be described.

The following Equation (1) is a general transmission line equation related to voltage.

A and B are constants, and x is a position on the transmission line. α is an initial phase. β is a phase constant representing a phase lead per unit length.

$${V}_{( x )} = {Ae}^{- ( α + j β ) x} + {Be}^{( α + j β ) x} \qquad ( 1 )$$

The first term on the right side of Equation (1) represents the traveling wave, and the second term represents the reflected wave. In the embodiment, it is assumed that the impedance of the first signal line 121 matches the impedance of the phase detector electrically connected to the first signal line. In the transmission line, an unnecessary wave is not generated at the connection point of the first signal line 121 and the phase detector, and the second term is zero. Hence, the transmission line equation in the case of the first signal line 121 of the embodiment can be expressed by the following Equation (2).

$${V}_{( x )} = {Ae}^{- ( α + j β ) x} \qquad ( 2 )$$

The phase constant β can be modified as in the following Equation (3). L is the inductor of a circuit model equivalent to the transmission line, C is the capacitance of the circuit model equivalent to the transmission line, εγ_eff is the effective dielectric constant, ε0 is the dielectric constant of vacuum, μ0 is the permeability of vacuum, and c is the speed of light.

$$β = ω \sqrt{LC} = ω \sqrt{{ε}_{r\_eff} {ε}_{0} {μ}_{0}} = \frac{ω \sqrt{{ε}_{r\_eff}}}{c} \qquad ( 3 )$$

It can be interpreted from Equation (3) that the larger the effective dielectric constant εγ_eff, the larger the phase constant β. That is, the larger the effective dielectric constant εγ_eff, the larger the phase lead per unit length.

As described above, when the skin 201 is pressed against the first signal line 121, the electric field vector E2 passes through the skin 201. Hence, the lead of the phase of the AC signal passing through the first signal line 121 changes according to the dielectric constant of the skin 201.

When the subject has a meal, the blood glucose level rises, and the glucose concentration in the interstitial fluid of the dermis layer rises. Moreover, in a specific frequency range (for example, a range of frequencies higher than the inflection point 300 of FIG. 1), the higher the glucose concentration, the higher the dielectric constant. Hence, when the blood glucose level of the subject rises, the phase of the AC signal passing through the first signal line 121 leads.

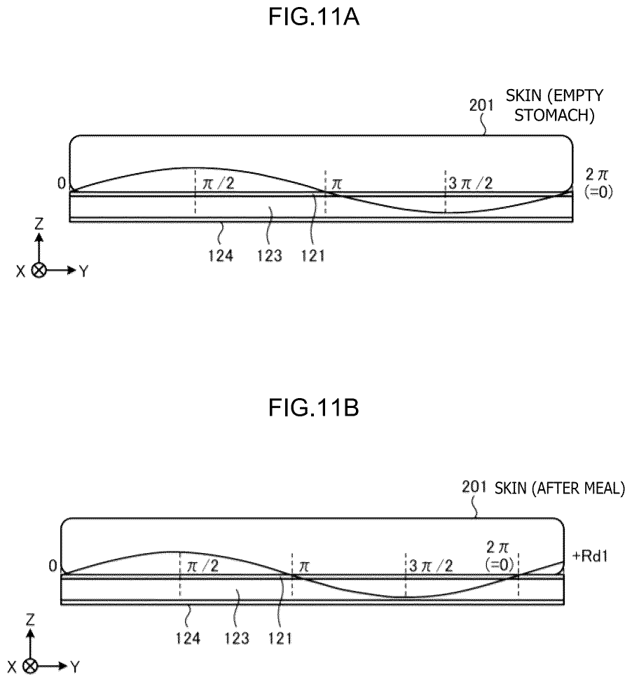

FIG. 11A describes the phase change when the stomach is empty, and FIG. 11B describes the phase change after the meal. FIGS. 11A and 11B describe changes in the phase of the AC signal passing through the first signal line 121 when the skin 201 of the subject is pressed against the first signal line 121 of the sensor 12.

To facilitate the understanding, it is assumed here that, when the stomach of the subject is empty, the wavelength of the AC signal at the dielectric constant of the skin 201 with the empty stomach is equal to the length (length in the Y direction here) from the input end (left end) to the output end (right end) of the first signal line 121.

That is, when the subject with the empty stomach touches the first signal line 121, the AC signal is transmitted at the wavelength equal to the length of the first signal line 121 as illustrated in FIG. 11A. Hence, when the phase of the AC signal at the input end of the first signal line 121 is 0 radians, the phase of the AC signal at the output end of the first signal line 121 is 0 radians.

When the subject touches the first signal line 121 in a state in which the blood glucose level has risen after the meal, the wavelength becomes short as illustrated in FIG. 11B. When the phase of the AC signal input to one end of the first signal line 121 is 0 radians, the phase of the AC signal output from the other end of the first signal line 121 leads according to the reduction in the wavelength, compared to the phase illustrated in FIG. 11A. The phase lead in the AC signal passing through the first signal line 121 in reference to the case where the stomach is empty will be referred to as a phase lead Rd1.

In the measurement of the blood glucose level, the skin 201 of the subject is pressed against the surface 123a, and the sensor 12 (substrate) comes under pressure. In a case where, for example, the sensor 12 is implemented in the smartwatch 1 illustrated in FIGS. 2 to 4, the skin 201 of the subject is pressed against the first signal line 121 when the subject tightens the band 3, and the pressure is applied to the sensor 12.

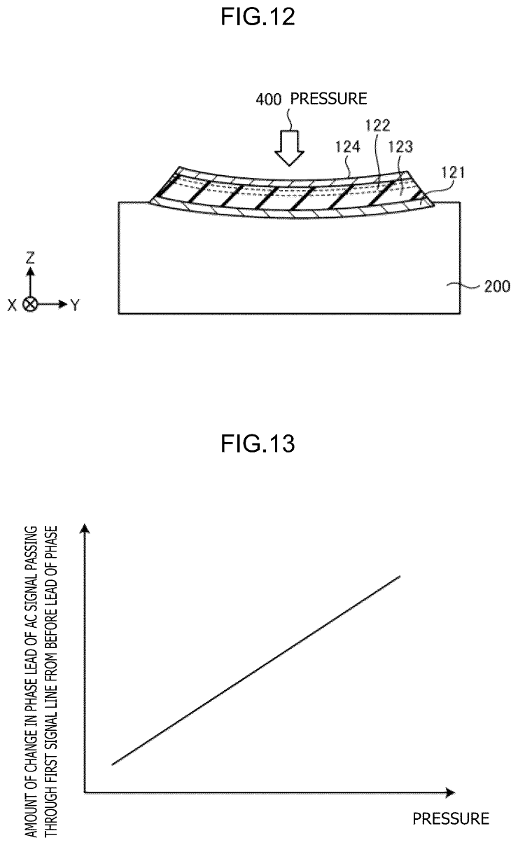

FIG. 12 is a diagram describing the application of the pressure to the sensor 12 when the subject tightens the band 3.

As illustrated in FIG. 12, when the band 3 is tightened, a strong pressure 400 makes the neighborhood of the center of the sensor 12, that is, the center of the first signal line 121, project downward, and the first signal line 121 is warped.

The pressure 400 affects the constituent elements of the sensor 12 including the substrate 123 and the first signal line 121. The pressure 400 changes, for example, the shape or the internal stress of the substrate 123 or the first signal line 121, thereby changing the passage characteristics of the electromagnetic field distribution of the sensor 12. As a result, the phase of the AC signal passing through the first signal line 121 changes, and this reduces the accuracy of the blood glucose level measurement.

For example, the first signal line 121 is transformed by the pressure 400 such that the length in the Y direction becomes slightly longer. Due to the increase in the length of the first signal line 121, the phase of the AC signal at the output end of the first signal line 121 leads.

FIG. 13 is a diagram illustrating that the phase of the AC signal at the output end of the first signal line 121 changes according to the pressure 400.

As a result of measurement and verification using the sensor 12 of the embodiment, the inventor has found out that the phase difference tends to linearly change with respect to the change in pressure as illustrated in FIG. 13. In FIG. 13, a vertical axis indicates the amount of change (phase difference) in the phase lead of the AC signal passing through the first signal line 121 from before the lead of the phase when the AC signal is applied to the first signal line 121 in the case where the stomach is empty. A horizontal axis indicates the magnitude of the pressure 400. The pressure 400 is applied in the −Z direction at the center of the sensor 12 in the Y direction. A commercially available pressure sensor is provided on the substrate to measure the pressure. In the example illustrated in FIG. 13, it can be interpreted that the larger the pressure 400, the more the phase of the AC signal leads. It can be assumed that the blood glucose level does not change when the stomach is empty, and hence, it can be stated that the phase difference has changed only by the influence of the change in the pressure.

It can be considered that the phase of the AC signal passing through the first signal line 121 changes not only by the transformation of the first signal line 121 due to the pressure 400, but also by the transformation of the substrate 123.

It can also be considered that the change in the internal stress of the first signal line 121 and the change in the internal stress of the substrate 123 caused by the pressure 400 change the phase.

In this way, the change in the phase of the AC signal can be described by a combination of factors. Hereinafter, the change in the phase of the AC signal passing through the signal line changed by the pressure 400 will be referred to as a “phase change caused by pressure.”

As described with reference to FIG. 13, the phase lead Rd1 acquired from the first signal line 121 reflects not only the change in the blood glucose level of the subject, but also the “phase change caused by pressure” at the measurement. Hence, the accuracy of the measured value is reduced if the measured value of the blood glucose level is obtained only on the basis of the phase lead Rd1.

In the first embodiment, a second signal line 122 as a component that increases the measurement accuracy of the blood glucose level is provided on the substrate 123. In addition, the AC signal is input to both the first signal line 121 and the second signal line 122.

As illustrated in FIGS. 5 and 8, the second signal line 122 has the same straight line shape as the first signal line 121 and is provided on the sensor 12 such that the second signal line 122 is parallel to the first signal line 121. Hence, when the sensor 12 comes under the pressure 400, the second signal line 122 is transformed in the same way as the first signal line 121 as illustrated in FIG. 12, and it can be considered that the stress distribution of the second signal line 122 is similar to the stress distribution of the first signal line 121. As a result, it can be considered that the phase change caused by pressure of the AC signal flowing through the second signal line 122 is a value close to the phase change caused by pressure of the AC signal flowing through the first signal line 121.

Moreover, any one of the width, the thickness, and the length of the second signal line 122 may be the same as that of the first signal line 121. In this way, the amounts of transformation or the stress distributions of the second signal line 122 and the first signal line 121 can be closer to each other when the sensor 12 comes under the pressure 400. As described later, the widths, the thicknesses, and the lengths of the second signal line 122 and the first signal line 121 may not be the same if the characteristic impedances of the second signal line 122 and the first signal line 121 can be brought into line with each other. The phase lead may vary if none of the widths, the thicknesses, and the lengths of the second signal line 122 and the first signal line 121 are the same. In that case, the amount of change can be stored, and a computation circuit can compensate the change at the measurement.

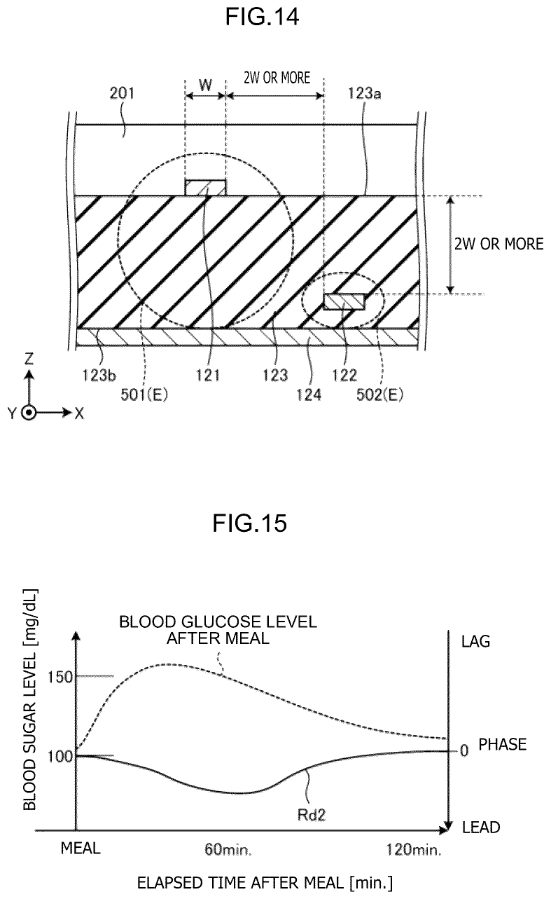

Further, mutual interference (crosstalk) of electromagnetic waves occurs depending on the arrangement of the first signal line 121 and the second signal line 122. Hence, as illustrated in FIG. 14, the second signal line 122 is separated from the first signal line 121 in the thickness direction of the substrate 123 to suppress the mutual interference. Hence, the second signal line 122 is embedded on the lower right of the first signal line 121. This is to prevent the second signal line 122 from overlapping the first signal line 121, separate them to the left and right, and separate the second signal line 122 downward from the surface of the substrate.

FIG. 14 is a diagram illustrating a positional relation between the first signal line 121 and the second signal line 122 provided on the sensor 12. Note that FIG. 14 illustrates a cross section in which the sensor 12 pressed against the skin 201 is cut along the XZ plane.

A 3 W rule is generally known. In the 3 W rule, the signal line group is arranged at such a pitch that the distance from the center to center of two signal lines is equal to or greater than three times the width of the signal lines, thereby suppressing the mutual interference of signals between the signal lines.

According to the 3 W rule, a space equal to or greater than 2 W can be provided between the first signal line 121 and the second signal line 122 as illustrated in FIG. 14 where W is the width of the first signal line 121, and the distance from the center of the wiring is added to obtain a pitch of equal to or greater than 3 W.

The 3 W rule also holds in the vertical direction when a space equal to or greater than 2 W is provided between the upper surface of the second signal line 122 and the surface 123a of the substrate 123. Hence, the mutual interference of the first signal line 121 and the second signal line 122 is suppressed to some extent.

As illustrated in FIG. 14, a range 501 indicates a range in which the intensity of the electric field generated from the first signal line 121 exceeds a predetermined level, and a range 502 indicates a range in which the intensity of the electric field generated from the second signal line 122 exceeds the predetermined level.

The predetermined level described here is an electric field level for suppressing the mutual interference. By arranging them according to the 3 W rule, the intensity of the electric field does not exceed the predetermined level, and the mutual interference can be suppressed.

In addition, to keep the range 502 away downward from the surface 123a, the second signal line 122 can be provided such that, for example, the distance from the surface 123a is 2 W. As a result, the second signal line 122 is close to the ground conductor, and this can suppress the influence of the electric field.

Note that the closer the signal line from the ground conductor, the lower the characteristic impedance of the signal line tends to be. The narrower the width of the signal line, the higher the characteristic impedance of the signal line tends to be. Furthermore, the thinner the thickness of the signal line, the higher the characteristic impedance of the signal line tends to be.

Further, the second signal line 122 in this example is arranged at a position closer to the ground conductor 124 than to the first signal line 121. Hence, to make the characteristic impedance of the first signal line 121 equal to the characteristic impedance of the second signal line 122, the designer may make the width of the second signal line 122 narrower than the width of the first signal line 121 or make the thickness of the second signal line 122 thinner than the thickness of the first signal line 121.

The same AC signal as the AC signal input to the input end of the first signal line 121 is input to the input end of the second signal line 122. The blood glucose level measurement apparatus 1 computes the blood glucose level on the basis of the phase difference between the AC signal passing through the second signal line 122 and the AC signal passing through the first signal line 121.

The phase difference of the AC signal passing through the first signal line 121 with respect to the AC signal passing through the second signal line 122 will be referred to as Rx. The phase difference Rx when the stomach is empty will be referred to as an empty stomach phase difference Ri, and the lead of the phase difference Rx from the empty stomach phase difference Ri will be referred to as Rd2.

The first signal line 121 and the second signal line 122 are arranged as described above, and this suppresses the phase change in the phase difference Rx caused by the applied pressure. Hence, unlike the phase lead Rd1, the phase lead Rd2 changes according to the blood glucose level of the subject while the influence of the applied pressure is suppressed.

FIG. 15 is a diagram describing a time shift of the phase lead Rd2 when the first signal line 121 is pressed against the skin 201 of the subject. In FIG. 15, a horizontal axis indicates the elapsed time after the meal. A vertical axis on the left indicates the blood glucose level, and the vertical axis on the right indicates the phase.

As illustrated in FIG. 15, once the subject has a meal, the blood glucose level of the subject starts to rise compared to when the stomach is empty. Consequently, the phase lead Rd2 increases according to the rise in the blood glucose level. In addition, when the blood glucose level shifts from rise to fall, the phase lead Rd2 shifts from increase to decrease. In this way, the phase lead Rd2 changes in conjunction with the change in the blood glucose level with respect to the blood glucose level with empty stomach.

The blood glucose level measurement apparatus 1 calculates the phase lead Rd2 and calculates the measured value of the blood glucose level on the basis of the phase lead Rd2.

FIG. 5 will be further described.

The oscillation circuit 11 produces an AC signal with a single frequency. The frequency of the AC signal produced by the oscillation circuit 11 is a frequency selected from a range that largely changes according to the change in the dielectric constant of the skin changed according to the blood glucose level. The oscillation circuit 11 produces, for example, an AC signal with a frequency selected from the range 310 of FIG. 1. Note that the frequency of the AC signal produced by the oscillation circuit 11 may be selected from a range other than the range 310.

The transmission path of the AC signal connected to the oscillation circuit 11 is branched into two. One is connected to the input end of the first signal line 121, and the other is connected to the input end of the second signal line 122.

The output end of the first signal line 121 is connected to the phase detector 13. In addition, the output end of the second signal line 122 is connected to the phase detector 13. Hence, the AC signal passing through the first signal line 121 and the AC signal passing through the second signal line 122 are input to the phase detector 13. The AC signal passing through the first signal line 121 and input to the phase detector 13 will be referred to as a measurement signal. The AC signal passing through the second signal line 122 and input to the phase detector 13 will be referred to as a reference signal.

Note that the AC signal produced by the oscillation circuit 11 is an example of a first signal. The measurement signal is an example of a second signal. The reference signal is an example of a third signal.

The phase detector 13 detects the phase difference Rx between the measurement signal and the reference signal and inputs the detected value of the phase difference Rx to the computation circuit 14. The phase detector 13 may also be called a phase comparator.

The phase of the measurement signal is changed according to the influence of the blood glucose level of the subject and the pressure, and the phase of the reference signal is changed according to the phase change caused by the pressure while the phase change caused by the change in the blood glucose level of the subject is suppressed. Hence, the phase detector 13 detects, as the phase difference Rx, the phase difference changed by the influence of the blood glucose level of the subject while the phase change caused by the applied pressure is suppressed.

The computation circuit 14 is a processor that executes a predetermined computation process. The computation circuit 14 is, for example, a microcomputer unit including a CPU (Central Processing Unit) and a memory storing a program, and the CPU executes the computation process according to the program. Note that the computation circuit 14 may include a hardware circuit, such as FPGA (Field-Programmable Gate Array) or ASIC (Application Specific Integrated Circuit).

The computation circuit 14 acquires the measured value of the blood glucose level of the subject on the basis of the phase difference Rx input from the phase detector 13.

The computation circuit 14 outputs the measured value of the blood glucose level to the display apparatus 15. Note that the output method of the measured value of the blood glucose level by the computation circuit 14 is not limited to this. For example, in a case in which the blood glucose level measurement apparatus 1 includes a printing apparatus, a speaker, or other devices, the computation circuit 14 may output the measured value of the blood glucose level to the printing apparatus, the speaker, or other devices. In a case in which the blood glucose level measurement apparatus 1 includes a memory, the measured value of the blood glucose level may be output to the memory. In a case in which the blood glucose level measurement apparatus 1 includes a communication apparatus, the computation circuit 14 may output the measured value of the blood glucose level to an external apparatus through the communication apparatus.

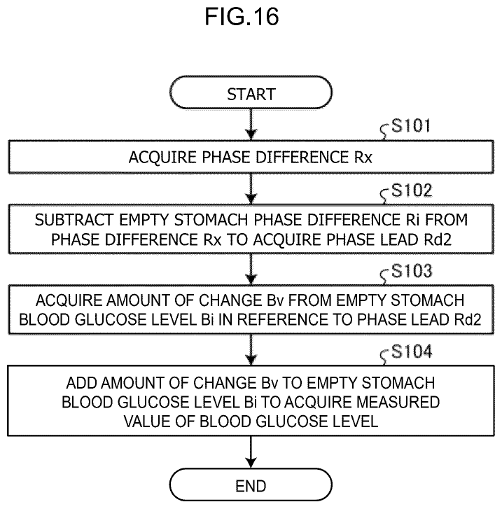

FIG. 16 is a flow chart illustrating an example of an operation of the blood glucose level measurement apparatus 1 according to the first embodiment. The series of operations illustrated in FIG. 16 are executed in a state in which the skin of the subject touches the first signal line 121 to measure the blood glucose level.

The phase detector 13 acquires the phase difference Rx between the measurement signal and the reference signal (S101). The phase difference Rx is input to the computation circuit 14.

The computation circuit 14 subtracts the empty stomach phase difference Ri, which is the phase difference Rx between the sensor passage signal and the local signal in the state in which the stomach of the subject is empty, from the phase difference Rx obtained in S101 to acquire the phase lead Rd2 (S102).

It is assumed that the empty stomach phase difference Ri is measured in advance and stored in the computation circuit 14 or a memory that can be accessed by the computation circuit 14. For example, in a case in which the blood glucose level measurement apparatus 1 is implemented in a wearable apparatus, the subject wears the blood glucose level measurement apparatus 1 all day long, and the computation circuit 14 stores the shift of the phase difference Rx in the period in which the subject wears the blood glucose level measurement apparatus 1. Moreover, the computation circuit 14 stores the lowest value of the phase difference Rx as the empty stomach phase difference Ri. Note that the acquisition method of the empty stomach phase difference Ri is not limited to this.

In addition, as with the empty stomach phase difference Ri, it is assumed that an empty stomach blood glucose level Bi that is the blood glucose level in the state in which the stomach of the subject is empty is measured in advance and stored in association with the empty stomach phase difference Ri in the computation circuit 14 or the memory that can be accessed by the computation circuit 14. The measurement method of the empty stomach blood glucose level Bi is not limited to a specific method. The empty stomach blood glucose level Bi can be measured by, for example, blood collection.

Following S102, the computation circuit 14 acquires an amount of change By in the blood glucose level from the empty stomach blood glucose level Bi on the basis of the phase lead Rd2 (S103).

For example, a calibration curve (referred to as a first calibration curve) representing the relation between the phase lead Rd2 and the amount of change By is acquired in advance by a simulation or an experiment with one or more subjects. The first calibration curve may be a function or may be information in a table format. The first calibration curve is stored in advance in the computation circuit 14 or the memory that can be accessed by the computation circuit 14. In S103, the computation circuit 14 acquires the amount of change By at the time of the execution of S103 on the basis of the phase lead Rd2 acquired in S102 and the first calibration curve.

Following S103, the computation circuit 14 adds the amount of change By acquired in S103 to the empty stomach blood glucose level Bi to acquire the measured value of the blood glucose level (S104). Then, the operation of the blood glucose level measurement apparatus 1 ends.

Note that the operation for acquiring the measurement value of the blood glucose level illustrated in FIG. 16 is just an example. The operation for acquiring the measured value of the blood glucose level can be modified in various ways.

For example, a calibration curve (referred to as a second calibration curve) representing the relation between the phase difference Rx and the blood glucose level is acquired in advance by a simulation or an experiment with one or more subjects and stored in advance in the computation circuit 14 or the memory that can be accessed by the computation circuit 14. Moreover, the computation circuit 14 may acquire the measured value of the blood glucose level on the basis of the phase difference Rx acquired in S101 and the second calibration curve.

Alternatively, the computation circuit 14 may calculate a dielectric constant εx of the skin in reference to the phase difference Rx and acquire the measured value of the blood glucose level in reference to the dielectric constant εx of the skin. For example, the computation circuit 14 converts the phase difference Rx into the dielectric constant εx of the skin on the basis of, for example, the following Equation (4). In Equation (4), a and b are coefficients obtained according to the relation between the dielectric constant and the phase difference Rx acquired in advance by, for example, pressing a sample with a known dielectric constant against the first signal line 121 to acquire the phase difference Rx.

$${ε}_{x} = a \times Rx + b \qquad ( 4 )$$

Further, the computation circuit 14 acquires the measured value of the blood glucose level in reference to the dielectric constant εx of the skin. For example, a calibration curve (referred to as a third calibration curve) representing the relation between the dielectric constant Ex of the skin and the blood glucose level is acquired in advance by a simulation or an experiment with one or more subjects and stored in advance in the computation circuit 14 or the memory that can be accessed by the computation circuit 14. The computation circuit 14 acquires the measured value of the blood glucose level in reference to the dielectric constant εx of the skin acquired from Equation (4) and the third calibration curve.

Note that the computation circuit 14 calculates the measured value of the blood glucose level in reference to the empty stomach blood glucose level Bi of the subject in the operation illustrated in FIG. 16 because the wavelength of the AC signal transmitted through the first signal line 121 may vary depending on the race, the sex, the individual differences in body composition, and other parameters of the subject even if the blood glucose level is the same. The empty stomach phase difference Ri and the empty stomach blood glucose level Bi of the subject are acquired in advance, and the measured value of the blood glucose level is calculated in reference to them. Hence, the blood glucose level can accurately be measured even when the race, the sex, the individual differences in body composition, and other parameters of the subject vary.

Another example of the operation in consideration of the race, the sex, the individual differences in body composition, and other parameters of the subject includes an operation described next. For example, a glucose tolerance test is carried out for the subject, and a calibration curve (referred to as a fourth calibration curve) representing the relation between the phase difference Rx and the blood glucose level obtained by blood collection or another optional blood glucose level measurement apparatus is created in the glucose tolerance test. The fourth calibration curve is stored in the computation circuit 14 or the memory that can be accessed by the computation circuit 14. Once the phase difference Rx obtained in S101 is input, the computation circuit 14 acquires the measured value of the blood glucose level on the basis of the phase difference Rx and the fourth calibration curve. According to the example of the operation, the fourth calibration curve created for each subject is used, and the blood glucose level can accurately be measured even in a case in which the race, the sex, the individual differences in body composition, and other parameters of the subject vary.

Even in a case in which the race, the sex, the individual differences in body composition, and other parameters of the subject are taken into account, the computation circuit 14 may calculate the dielectric constant cx of the skin in reference to the phase difference Rx and acquire the measured value of the blood glucose level in reference to the dielectric constant εx of the skin.

In this way, according to the first embodiment, the blood glucose level measurement apparatus 1 includes the substrate 123 that is a dielectric, the ground conductor 124 provided on the substrate 123, the first signal line 121 provided on the substrate 123 and against which a living body is pressed, the second signal line 122 provided on the substrate 123 such that the second signal line 122 is separated from the first signal line 121, and the second signal line 122 does not come into contact with the living body when the living body is pressed against the first signal line 121, the oscillation circuit 11 that oscillates an AC signal, a phase detector 13 that detects the phase difference RX between the measurement signal which is the AC signal passing through the first signal line 121, and the reference signal which is the AC signal passing through the second signal line 122, and the computation circuit 14 that acquires the measured value of the blood glucose level in reference to the phase difference Rx.

In addition, according to the first embodiment, the second signal line 122 has the same length and shape as the first signal line 121, and the second signal line 122 is provided parallel to the first signal line 121 on the substrate 123.

Hence, when the sensor 12 comes under the pressure 400, the phase change caused by pressure of the AC signal flowing through the second signal line 122 can be close to the phase change caused by pressure of the AC signal flowing through the first signal line 121. As a result, the influence of the pressure on the phase difference Rx can be suppressed, and this improves the accuracy of the measured value of the blood glucose level.

Note that the first signal line 121 and the second signal line 122 have straight line shapes in the description above. The shape of the first signal line 121 may not be the straight line. In addition, even if the second signal line 122 has a shape other than the straight line, the influence of the pressure on the phase difference Rx can be suppressed as long as the shape and the length of the second signal line 122 are the same as those of the first signal line 121.

Moreover, the second signal line 122 may have a different length from the first signal line 121. For example, the second signal line 122 may have the same shape as the first signal line 121 and a different length from the first signal line 121, and the computation circuit 14 may be configured to correct the phase change caused by the difference between the lengths of the first signal line 121 and the second signal line 122.

In addition, according to the first embodiment, the substrate 123 includes the surface 123a, and the surface 123b that is a surface on the opposite side of the surface 123a. The first signal line 121 is provided on the surface 123a, and the ground conductor 124 with a flat shape is provided on the surface 123b. The second signal line 122 is embedded into the substrate 123 at a distance of equal to or greater than 2 W from the surface 123a, not overlapping the first signal line 121 in projection view in the Z direction, and at a distance of equal to or greater than 2 W from the first signal line 121.

Hence, the interaction of the AC signal passing through the first signal line 121 and the AC signal passing through the second signal line 122 can be suppressed, and the influence of the blood glucose level of the subject on the AC signal passing through the second signal line 122 can be suppressed.

### Second Embodiment

As described above, the larger the blood glucose level, the more the phase of the AC signal passing through the signal line leads. The lead of the phase of the AC signal is synonymous with shortening of the wavelength of the AC signal.

A blood glucose level measurement apparatus 1a of a second embodiment observes a change in the wavelength of the measurement signal as a change in the frequency and acquires the measured value of the blood glucose level in reference to the change in the frequency. Hereinafter, the blood glucose level measurement apparatus 1a of the second embodiment will be described. Note that matters similar or analogous to the matters of the first embodiment will not be described or will be simply described.

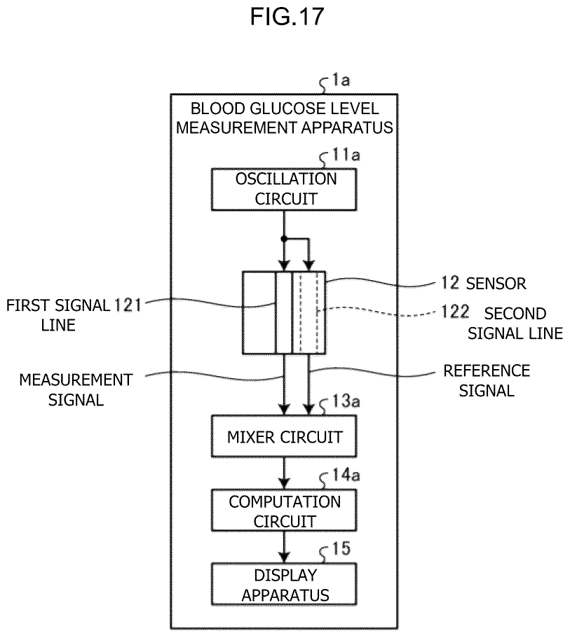

FIG. 17 is a schematic diagram illustrating an example of the configuration of the blood glucose level measurement apparatus 1a according to the second embodiment. As illustrated in FIG. 17, the blood glucose level measurement apparatus 1a includes an oscillation circuit 11a, the sensor 12, a mixer circuit 13a, a computation circuit 14a, and the display apparatus 15.

The oscillation circuit 11a oscillates an AC signal, that is, a chirp signal, in which the frequency temporally changes. The frequency band of the chirp signal oscillated by the oscillation circuit 11a is selected from a range in which the dielectric constant of the skin may change according to the blood glucose level. The oscillation circuit 11a oscillates, for example, a chirp signal in which the frequency changes in the frequency band selected from the range 310 of FIG. 1. Note that the frequency band of the chirp signal oscillated by the oscillation circuit 11a may be selected from a range other than the range 310.

The transmission path of the chirp signal connected to the oscillation circuit 11a is branched into two paths. One of the two branched transmission paths is connected to the input end of the first signal line 121, and the other of the two branched transmission paths is connected to the input end of the second signal line 122.

The output end of the first signal line 121 is connected to the mixer circuit 13a. In addition, the output end of the second signal line 122 is connected to the mixer circuit 13a. Hence, the chirp signal passing through the first signal line 121 and the chirp signal passing through the second signal line 122 are input to the mixer circuit 13a. Also in the second embodiment, the chirp signal passing through the first signal line 121 and input to the phase detector 13 will also be referred to as a measurement signal. The chirp signal passing through the second signal line 122 and input to the phase detector 13 will be referred to as a reference signal.

The mixer circuit 13a generates a beat frequency signal indicating the frequency difference between the measurement signal and the reference signal and inputs the beat frequency signal to the computation circuit 14a.

The computation circuit 14a uses the beat frequency signal in place of the phase difference Rx used by the computation circuit 14 of the first embodiment to acquire the measured value of the blood glucose level. The computation circuit 14a can use a freely selected method to output the acquired measured value of the blood glucose level, as with the computation circuit 14 of the first embodiment.

In this way, according to the second embodiment, the blood glucose level measurement apparatus 1a includes the mixer circuit 13a that outputs the beat frequency signal indicating the frequency difference between the sensor passage signal and the local signal, and the computation circuit 14a acquires the measured value of the blood glucose level in reference to the beat frequency signal.

Hence, the blood glucose level can be non-invasively and highly accurately measured as in the first embodiment.

### First Modification

The sensor 12 of the first embodiment and the second embodiment can be modified in various ways. A sensor 12-1 of a first modification described next can be applied in place of the sensor 12 of the first embodiment and the second embodiment.

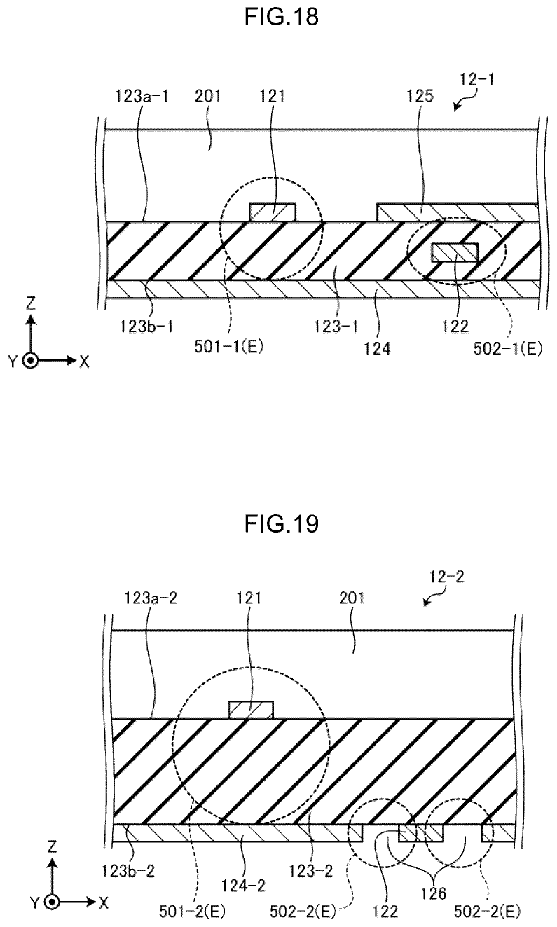

FIG. 18 is a cross-sectional view of the sensor 12-1 of the first modification cut along the XZ plane.

The sensor 12-1 includes a substrate 123-1. The first signal line 121 is provided on an upper surface 123a-1 of the substrate 123-1, and the skin 201 is pressed against the first signal line 121 in the blood glucose level measurement. The gapless ground conductor 124 is provided on the entire lower surface, which is a lower surface 123b-1 in FIG. 18, on the opposite side of the upper surface.

The second signal line 122 is embedded into the substrate 123 at a position not overlapping the first signal line 121 in projection view in the Z direction. In the example, the position not overlapping the first signal line 121 in projection view in the Z direction is a position separated from the first signal line 121 in the X direction.

The details are the same as the positional relation of FIG. 14.

Note that the first signal line 121 is a signal line with a straight line shape extending in the Y direction. The second signal line 122 has a straight line shape extending in the Y direction with the same length as the first signal line 121, and the second signal line 122 is provided parallel to the first signal line 121.

A second ground conductor 125 is provided on the surface 123a-1 in projection view in the Z direction so as to cover a region overlapping the second signal line 122, that is, the entire region. The second ground conductor 125 includes, for example, a material similar to the ground conductor 124.

In this way, the second signal line 122 is provided as a strip line placed between the ground conductor 124 and the second ground conductor 125.

Describing again, the first signal line is provided on one surface of the substrate from one side to another side, at the center of the substrate. In contrast, the second signal line is embedded into the substrate parallel to the first signal line, at a position not overlapping the first signal line in plan view. Moreover, the ground conductor 124 is provided on the entire other surface of the substrate. In addition, the second ground conductor 125 is provided without a gap on one surface of the substrate, from one side to another side of the substrate so as to cover the second signal line. In FIG. 18, the second ground conductor 125 covers approximately ⅓ to ½ of the substrate.

In the blood glucose level measurement, the skin 201 is pressed against the first signal line 121 provided on the surface 123a-1. In this case, the skin 201 may also come into contact with the second ground conductor 125 provided on the surface 123a-1. The second ground conductor 125 prevents the electric field vector generated from the second signal line 122 from entering the skin 201.

That is, the distance between the second signal line 122 and the surface 123a-1 in the vertical direction may be less than 2 W. Hence, the thickness of the substrate 123-1 can be smaller than the substrate 123.

501-1 represents a range in which the intensity of the electric field generated from the first signal line 121 exceeds the predetermined level, and 502-1 represents a range in which the intensity of the electric field generated from the second signal line 122 exceeds the predetermined level.

As illustrated in FIG. 18, even when the substrate 123-1 is formed with a thickness smaller than the substrate 123, the range 502-1 is absorbed by the second ground conductor 125 and does not reach the skin 201. In addition, the mutual interference of the first signal line 121 and the second signal line 122 is also suppressed by the presence of two ground conductors.

### Second Modification

FIG. 19 is a cross-sectional view of a sensor 12-2 of a second modification cut along the XZ plane. The sensor 12-2 includes a substrate 123-2. The first signal line 121 is provided on one surface 123a-2 of the substrate 123-2, and the first signal line 121 is pressed against the skin 201 in the blood glucose level measurement.

The second signal line 122 is provided on a surface 123b-2 on the opposite side of one surface, at a position not overlapping the first signal line 121 in projection view in the Z direction. In the example, the position not overlapping the first signal line 121 in projection view in the Z direction is a position separated from the first signal line 121 in the X direction.

Note that the first signal line 121 is a signal line with a straight line shape extending in the Y direction. The second signal line 122 has a straight line shape extending in the Y direction with the same length as the first signal line 121, and the second signal line 122 is provided parallel to the first signal line 121.

A ground conductor 124-2 is provided without a gap on substantially the entire surface 123b-2. Moreover, the second signal line 122 is provided parallel to the first signal line 121 without overlapping the first signal line 121.

Here, gapless metal Cu is patterned and formed by photo etching, and hence, grooves formed by removing the metal are formed on both sides of the second signal line.

The second signal line 122 is placed between the walls on both sides of the grooves of the ground conductor 124-2. That is, when the second signal line 122 is viewed, the ground conductor 124-2 is provided on both sides of the signal line. Hence, the second signal line 122 is provided as a coplanar line.

In a case in which the second signal line 122 is provided as a coplanar line, the second signal line 122 does not have to be embedded. Hence, the substrate 123 can be realized by a one-layer substrate with a double-sided conductor, instead of a multi-layer substrate. The thickness of the substrate 123 can be made small, and this is advantageous in terms of cost.

501-2 represents a range in which the intensity of the electric field generated from the first signal line 121 exceeds the predetermined level, and 502-2 represents ranges in which the intensity of the electric field generated from the second signal line 122 exceeds the predetermined level.

As illustrated in FIG. 19, there are ground conductors on both sides of the second signal line, and the magnetic field line is absorbed by the metals. This can suppress the electric field vector generated from the second signal line from reaching the skin.

Hence, the mutual interference of the first signal line 121 and the second signal line 122 is suppressed.

### Third Modification

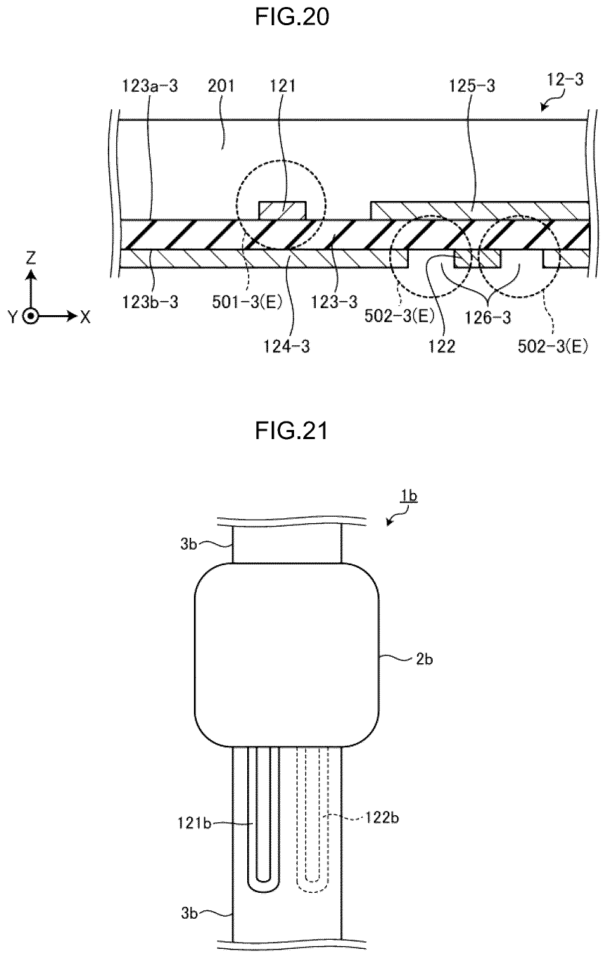

FIG. 20 is a cross-sectional view of a sensor 12-3 of a third modification cut along the XZ plane.

The sensor 12-3 includes a substrate 123-3. The first signal line 121 is provided on an upper surface 123a-3 of the substrate 123-3, and the skin 201 is pressed against the first signal line 121 in the blood glucose level measurement.

The second signal line 122 is provided on a surface 123b-3 on the opposite side of the upper surface 123a-3 of the substrate 123-3, at a position not overlapping the first signal line 121 in projection view in the Z direction. In the example, the position not overlapping the first signal line 121 in plan view in the Z direction is a position separated from the first signal line 121 in the X direction.

Note that the first signal line 121 is a signal line with a straight line shape extending in the Y direction. The second signal line 122 has a straight line shape extending in the Y direction with the same length as the first signal line 121, and the second signal line 122 is provided parallel to the first signal line 121.

Note that the lengths and the shapes of the first signal line 121 and the second signal line 122 can be modified similarly to the constituent elements with the same names described in the first embodiment.

A ground conductor 124-3 with a flat shape including grooves that expose the substrate are further provided on the surface 123b-3. The grooves are provided in the ground conductor 124-3 such that walls on both sides of the grooves sandwich the second signal line 122 with spaces 126-3.

In addition, a second ground conductor 125-3 is provided in a region overlapping the second signal line 122 on the surface 123a-3 in projection view in the Z direction. The second ground conductor 125-3 includes, for example, a material similar to the ground conductor 124-3.

That is, the second signal line 122 is provided as a grounded coplanar line.

The second ground conductor 125-3 prevents the electric field vector formed by the AC signal passing through the second signal line 122 from entering the skin 201. That is, even when the distance between the second signal line 122 and the surface 123a-3 is less than 2 W, the influence of the blood glucose level of the subject received by the AC signal passing through the second signal line 122 can be suppressed. Hence, the thickness of the substrate 123-3 can be made smaller than the substrate 123.

501-3 represents a range in which the intensity of the electric field formed by the AC signal passing through the first signal line 121 exceeds the predetermined level, and 502-3 represents ranges in which the intensity of the electric field formed by the AC signal passing through the second signal line 122 exceeds the predetermined level. As illustrated in FIG. 20, the entrance of the ranges 502-3 into the skin 201 is prevented. In addition, the range 501-3 and the ranges 502-3 are separated, and this suppresses the interaction of the AC signal passing through the first signal line 121 and the AC signal passing through the second signal line 122.

### Fourth Modification

The sensor is provided on the lower surface of the case 2 in the embodiments so far.

Here, a sensor 12-4 may be provided on a band 3b as illustrated in FIG. 21.

FIG. 21 is a diagram depicting a blood glucose level measurement apparatus 1b as well as the band 3b.

As in FIG. 21, the sensor 12-4 is provided on an abutting surface on the band 3 side, instead of the lower surface of the case 2 which is the abutting surface on the case 2 side.

The first signal line 121 is provided on the surface of the band 3 such that the first signal line 121 comes into contact with the skin of the subject in such a manner that the first signal line 121 is pressed against the skin 201 of the subject when the subject wears the blood glucose level measurement apparatus 1b. The second signal line 122 is embedded into an inner layer of the band 3 such that the second signal line 122 does not come into contact with the skin 201 of the subject when the subject wears the blood glucose level measurement apparatus 1b, more precisely, such that the influence of the blood glucose level of the subject on the AC signal passing through the second signal line 122 is suppressed. Although the first signal line 121 and the second signal line 122 each have a U shape in the example, the shapes of the first signal line 121 and the second signal line 122 are not limited to this. In addition, the sensor 12-4 can be modified in various ways as in the first to third modifications.

The first signal line and the second signal line are provided on the belt, and hence, the length of each of the two signal lines can be made longer than that in a case in which they are provided on the case. The contact of the measurement target with the skin increases, and this improves the measurement accuracy. Note that this is true when the contact of the belt and the skin is proper.

### Fifth Modification

In the examples described above, the blood glucose level measurement apparatuses 1, 1a, and 1b are watch-type wearable apparatuses. The mode of implementing the blood glucose level measurement apparatuses 1, 1a, and 1b is not limited to this. For example, the blood glucose level measurement apparatuses 1, 1a, and 1b may be fixed to the skin 201 by a tape or other fixing member, from above the case 2.

In addition, the first signal line 121 is provided on the part, against which the skin 201 is pressed, on the substrates 123, 123-1, 123-2, and 123-4 in the examples described above. The first signal line 121 may not necessarily be exposed as long as the electric field vector formed by the AC signal passing through the first signal line 121 enters the skin 201 in the blood glucose level measurement.

For example, a part including the first signal line 121 or all of the surfaces 123a, 123a-1, 123a-2, and 123a-3 provided with the first signal line 121 may be covered by a thin film having an insulating property. The thin film may include, for example, a solder resist or insulating ceramic such as silicon oxide. In this way, the living body is pressed against the first signal line 121 through the thin film, and this can prevent the breakage or corrosion of the first signal line 121 caused by the subject touching the first signal line 121.

The measurement apparatus that measures the blood glucose level as biological information has been described in the first embodiment, the second embodiment, and their modifications. Biological information other than the blood glucose level may be measured.

For example, the dielectric constant of the skin may be the biological information to be measured. The measurement apparatus of the embodiments may acquire the dielectric constant of the skin in reference to the phase difference or the frequency difference between the measurement signal and the reference signal and output the acquired dielectric constant of the skin.

In addition, the dielectric constant of the skin may also be affected by the amount of cancer cells. Hence, the amount of cancer cells may be the biological information to be measured in the measurement apparatus of the embodiments. The measurement apparatus of the embodiments may acquire the amount of cancer cells in reference to the phase difference or the frequency difference between the measurement signal and the reference signal and output the acquired amount of cancer cells.

As described in the first embodiment, the second embodiment, and their modifications, the measurement apparatus includes the substrate (for example, the substrates 123, 123-1, 123-2, and 123-3) that is a dielectric, the first signal line (for example, the first signal line 121) provided on the substrate and against which a living body is pressed, the second signal line (for example, the second signal line 122) provided on the substrate such that the second signal line is separated from the first signal line, and the second signal line does not come into contact with the living body when the living body is pressed against the first signal line, the oscillation circuit (for example, the oscillation circuits 11 and 11a) that oscillates an AC first signal, and a computation circuit (for example, the computation circuits 14 and 14a) that acquires the biological information according to the comparison of a second signal (for example, the measurement signal) that is the first signal passing through the first signal line and a third signal (for example, the reference signal) that is the first signal passing through the second signal line. The comparison of the AC signal passing through the first signal line and the AC signal not passing through the first signal line is the detection of the phase difference Rx in the first embodiment and is the detection of the frequency difference in the second embodiment.

Hence, the biological information can be non-invasively and highly accurately measured.

In addition, the measurement apparatus described in the first embodiment, the second embodiment, and their modifications may be a dielectric constant measurement apparatus that measures the dielectric constant of the skin. Modes of the measurement apparatus and the dielectric constant measurement apparatus are, for example, as follows.

### Note 1

A measurement apparatus including:
a substrate that is a dielectric;
a ground conductor provided on the substrate;
a first signal line provided separately from the ground conductor on the substrate and against which a living body is pressed;
a second signal line provided on the substrate such that the second signal line is separated from the ground conductor and the first signal line, and the second signal line does not come into contact with the living body when the living body is pressed against the first signal line;
an oscillation circuit that oscillates an AC first signal; and
a computation circuit that acquires biological information according to comparison of a second signal that is the first signal passing through the first signal line and a third signal that is the first signal passing through the second signal line.

### Note 2

The measurement apparatus according to Note 1, in which
the first signal line is linearly formed in plan view, and the second signal line is linearly formed and provided parallel to the first signal line on the substrate in plan view.

### Note 3

The measurement apparatus according to Note 1 or Note 2, in which
widths, thicknesses, or lengths of the first signal line and the second signal line are the same.

### Note 4

The measurement apparatus according to Note 1 or Note 2, in which
the substrate includes a first surface and a second surface on an opposite side of the first surface,
the first signal line is provided on the first surface,
the ground conductor has a flat shape and is provided on the second surface, and
the second signal line is embedded into the substrate at a distance of equal to or greater than twice the width of the first signal line from the first surface, not overlapping the first signal line in projection view in a thickness direction of the substrate, and at a distance of equal to or greater than twice the width of the first signal line from the first signal line.

### Note 5

The measurement apparatus according to Note 1 or Note 2, in which
the substrate includes a first surface and a second surface on an opposite side of the first surface,
the first signal line is provided on the first surface,
the ground conductor has a flat shape and is provided on the second surface,
the second signal line is embedded into the substrate at a position not overlapping the first signal line in projection view in a thickness direction of the substrate, and
a second ground conductor is provided in a region overlapping the second signal line on the first surface in the projection view.

### Note 6

The measurement apparatus according to Note 1 or Note 2, in which
the substrate includes a first surface and a second surface on an opposite side of the first surface,
the first signal line is provided on the first surface,
the second signal line is provided at a position not overlapping the first signal line on the second surface in projection view in a thickness direction of the substrate, and
the ground conductor is provided on the second surface and has a flat shape including grooves that expose the substrate, walls on both sides of the grooves providing spaces and sandwiching the second signal line.

### Note 7

The measurement apparatus according to Note 1 or Note 2, in which
the substrate includes a first surface and a second surface on an opposite side of the first surface,
the first signal line is provided on the first surface,
the second signal line is provided at a position not overlapping the first signal line on the second surface in projection view in a thickness direction of the substrate,
the ground conductor is provided on the second surface and has a flat shape including grooves that expose the substrate, walls on both sides of the grooves providing spaces and sandwiching the second signal line, and
a second ground conductor is provided in a region overlapping the second signal line on the first surface in the projection view.

### Note 8

The measurement apparatus according to Note 1 or Note 2, further including:
a phase detector that detects a phase difference between the second signal and the third signal, wherein
the computation circuit acquires the biological information in reference to the phase difference.

### Note 9

The measurement apparatus according to Note 1 or Note 2, in which
the first signal is a chirp signal,
the measurement apparatus further includes a mixer circuit that receives the second signal and the third signal and that outputs a signal corresponding to a frequency difference between the second signal and the third signal, and
the computation circuit acquires the biological information in reference to the signal corresponding to the frequency difference.

### Note 10

The measurement apparatus according to Note 1 or Note 2, in which
the biological information includes a dielectric constant of the living body, a blood glucose level of the living body, or an amount of cancer cells of the living body.

### Note 11

A dielectric constant measurement apparatus of skin, including:
a rectangular printed board;
a strip that is a line for skin dielectric constant sensor including a conductive thin film provided across from a first side of a front surface of the printed board to a second side facing the first side;
a gapless film provided on a back surface of the printed board, covering the strip, provided up to four sides of the printed board, and including the conductive thin film; and
a strain sensor extending separately from the strip in plan view and embedded into the printed board.

### Note 12

The dielectric constant measurement apparatus of skin according to Note 11, in which
the strip includes a conductive pattern extended up to two facing sides, and the gapless film includes a same material as that of the conductive pattern.

### Note 13

The dielectric constant measurement apparatus of skin according to Note 11, in which
the strain sensor includes a same material as that of the strip and includes a conductive pattern extended up to two facing sides.

The embodiment has an advantageous effect that the measurement apparatus and the dielectric constant measurement apparatus that can non-invasively and highly accurately measure the biological information can be provided.

Although some embodiments of the present invention have been described, the embodiments are presented as examples, and the embodiments are not intended to limit the scope of the invention. These new embodiments can be carried out in various other modes, and the embodiments can be omitted, replaced, or changed in various ways without departing from the scope of the invention. These embodiments and modifications of the embodiments are included in the scope and subject matter of the invention and included in the scope of the inventions described in the claims and equivalents of the claims.

What is claimed is:

1. A measurement apparatus comprising:
a substrate that is a dielectric;
a ground conductor provided on the substrate;
a first signal line provided separately from the ground conductor on the substrate and against which a living body is pressed;
a second signal line provided on the substrate such that the second signal line is separated from the ground conductor and the first signal line, and the second signal line does not come into contact with the living body when the living body is pressed against the first signal line;
an oscillation circuit that oscillates an alternating current first signal; and
a computation circuit that acquires biological information according to comparison of a second signal that is the first signal passing through the first signal line and a third signal that is the first signal passing through the second signal line.

2. The measurement apparatus according to claim 1, wherein
the first signal line is linearly formed in plan view, and the second signal line is linearly formed and provided parallel to the first signal line on the substrate in plan view.

3. The measurement apparatus according to claim 1, wherein
widths, thicknesses, or lengths of the first signal line and the second signal line are the same.

4. The measurement apparatus according to claim 1, wherein
the substrate includes a first surface and a second surface on an opposite side of the first surface,
the first signal line is provided on the first surface,
the ground conductor has a flat shape and is provided on the second surface, and
the second signal line is embedded into the substrate at a distance of equal to or greater than twice the width of the first signal line from the first surface, not overlapping the first signal line in projection view in a thickness direction of the substrate, and at a distance of equal to or greater than twice the width of the first signal line from the first signal line.

5. The measurement apparatus according to claim 1, wherein
the substrate includes a first surface and a second surface on an opposite side of the first surface,
the first signal line is provided on the first surface,
the ground conductor has a flat shape and is provided on the second surface,
the second signal line is embedded into the substrate at a position not overlapping the first signal line in projection view in a thickness direction of the substrate, and
a second ground conductor is provided in a region overlapping the second signal line on the first surface in the projection view.

6. The measurement apparatus according to claim 1, wherein
the substrate includes a first surface and a second surface on an opposite side of the first surface,
the first signal line is provided on the first surface,
the second signal line is provided at a position not overlapping the first signal line on the second surface in projection view in a thickness direction of the substrate, and
the ground conductor is provided on the second surface and has a flat shape including grooves that expose the substrate, walls on both sides of the grooves providing spaces and sandwiching the second signal line.

7. The measurement apparatus according to claim 1, wherein
the substrate includes a first surface and a second surface on an opposite side of the first surface,
the first signal line is provided on the first surface,
the second signal line is provided at a position not overlapping the first signal line on the second surface in projection view in a thickness direction of the substrate,
the ground conductor is provided on the second surface and has a flat shape including grooves that expose the substrate, walls on both sides of the grooves providing spaces and sandwiching the second signal line, and
a second ground conductor is provided in a region overlapping the second signal line on the first surface in the projection view.

8. The measurement apparatus according to claim 1, further comprising:
a phase detector that detects a phase difference between the second signal and the third signal, wherein
the computation circuit acquires the biological information in reference to the phase difference.

9. The measurement apparatus according to claim 1, wherein
the first signal is a chirp signal,
the measurement apparatus further includes a mixer circuit that receives the second signal and the third signal and that outputs a signal corresponding to a frequency difference between the second signal and the third signal, and
the computation circuit acquires the biological information in reference to the signal corresponding to the frequency difference.

10. The measurement apparatus according to claim 1, wherein
the biological information includes a dielectric constant of the living body, a blood glucose level of the living body, or an amount of cancer cells of the living body.

11. A dielectric constant measurement apparatus of skin, comprising:
a rectangular printed board;
a strip that is a line for skin dielectric constant sensor including a conductive thin film provided across from a first side of a front surface of the printed board to a second side facing the first side;
a gapless film provided on a back surface of the printed board, covering the strip, provided up to four sides of the printed board, and including the conductive thin film; and
a strain sensor extending separately from the strip in plan view and embedded into the printed board.

12. The dielectric constant measurement apparatus of skin according to claim 11, wherein
the strip includes a conductive pattern extended up to two facing sides, and the gapless film includes a same material as that of the conductive pattern.

13. The dielectric constant measurement apparatus of skin according to claim 11, wherein
the strain sensor includes a same material as that of the strip and includes a conductive pattern extended up to two facing sides.
